(19) United States
(12) Patent Application Publication
(10) Pub. No.: US 2018/0340225 A1
(43) Pub. Date: Nov. 29, 2018

(54) INTESTINAL METAGENOMIC FEATURE AS SELECTION MARKER OF CURATIVE EFFECT OF ACARBOSE FOR TREATING TYPE 2 DIABETES

(71) Applicants: SHANGHAI INSTITUTE FOR ENDOCRINE AND METABOLIC DISEASES, Shanghai (CN); BGI Shenzhen, Shenzhen (CN)

(72) Inventors: Guang NING, Shanghai (CN); Dongya ZHANG, Shenzhen (CN); Weiqing WANG, Shanghai (CN); Xiaokai WANG, Shenzhen (CN); Yanyun GU, Shanghai (CN); Qiang FENG, Shenzhen (CN); Ruixin LIU, Shanghai (CN); Xiaoqiang XU, Shenzhen (CN); Huahui REN, Shenzhen (CN); Huanzi ZHONG, Shenzhen (CN)

(73) Assignees: SHANGHAI INSTITUTE FOR ENDOCRINE AND METABOLIC DISEASES, Shanghai (CN); BGI Shenzhen, Shenzhen (CN)

(21) Appl. No.: 15/771,398

(22) PCT Filed: Dec. 16, 2015
(86) PCT No.: PCT/CN2015/097566

§ 371 Date: Apr. 26, 2018

(30) Foreign Application Priority Data
Oct. 26, 2015 (CN) 201510703463.9

(51) Int. Cl. C12Q 1/6883

(52) U.S. Cl. CPC C12Q 1/6883; C12Q 2600/106

(57) ABSTRACT

An intestinal microorganism metagenomic feature is a bacteroid intestinal pattern. By using the intestinal metagenomic pattern, it was found that type 2 diabetic patients with different intestinal parasitic bacterial flora had remarkably different responses to treatment with the diabetic hypoglycemic drug acarbose. Therefore, before treatments, the intestinal patterns of the patients can be assessed to select those patients likely to experience optimal curative effects, and to determine whether acarbose is suitable for the treatment of individual patients with type 2 diabetes. In addition, while intestinal patterns are conventionally distinguished by DNA sequencing or PCT amplification of parasitic bacteria in excrements, the intestinal patterns can be well distinguished using a bile acid composition, especially secondary bile acids, under baseline conditions. The intestinal patterns can be identified through markers in blood, and then be used as markers for diagnosis.

### TECHNICAL FIELD

The present invention relates to use of the characteristics of gut microbiota metagenome as a screening marker for Acarbose efficacy in patients with Type 2 diabetes.

### BACKGROUND ART

At present, drug efficacy assessment and pre-treatment classification diagnosis are not available in the treatment of Type 2 diabetes. The pathological and physiological mechanisms of Type 2 diabetes mainly include insulin resistance and insulin secretion deficiency. Although there are drugs for insulin resistance and insulin secretion deficiency in the treatment of Type 2 diabetes, no scientific and feasible method is available to classify patients into mainly insulin resistance or mainly insulin secretion deficiency.

Currently, the clinically feasible program is to measure the patient BMI, waist circumference and insulin level. According to the BMI, waist circumference that exceed Chinese standard, or the HOMAIR calculated by patient's fasting blood glucose or insulin level, the insulin resistance can be judged. There are no universal standard for the insulin level at home and abroad. Generally, it is represented by the HOMA β index calculated by patient's blood glucose and insulin, but it cannot be used as an index for determining the degree of insulin resistance and insulin secretion deficiency. Therefore, it is unable to meet the requirements for precision medical care.

Clinically, the glucose clamp test is used to accurately assess the insulin resistance and β cell functions. The glucose clamp test with positive-glucose high insulin level is used to assess the insulin resistance levels, while the clamp test with high glucose level is used to assess the β cell insulin secretion functions. The two methods take long time, and patients need to lie in bed for 4 to 5 hours. The operations must be completed by experienced nurses. Blood should be collected from multiple points for the real-time monitoring of blood glucose and the determination of insulin level. This method is expensive, with poor patient compliance, so it is difficult to carry out clinically.

The precision medicine raises the requirement of individualized diagnosis and treatment. The tumor-targeted drugs have been used clinically. However, no effective regimen for targeted therapy of Type 2 diabetes has been found so far. There are a number of therapeutic regimens for the Type 2 diabetes and patients have different responses, which lead to a low blood glucose control rate for patients with Type 2 diabetes. For the main pathogenesis of Type 2 diabetes, there are a variety of drugs for insulin secretion deficiency and insulin resistance, but no simple and exact clinical diagnosis method for insulin secretion deficiency or insulin resistance is available.

The existing studies use the liver, fat (insulin resistance) and islet β cells (islet function) as the main organs involved in Type 2 diabetes. Recently, the pathological and physiological functions of gut microbiota and intestinal mucosal epithelial absorption, barrier and endocrine are increasingly recognized for the pathogenesis of Type 2 diabetes and treatment strategy. For example, gut microbiota metagenome studied have shown that there was significant difference in the gut microbiota between patients with Type 2 diabetes and normal patients [Qin, J., et al., A metagenome-wide association study of gut microbiota in Type 2 diabetes. Nature, 2012.]. The gut-modified bariatric surgery could reduce the body weight of obese patients, and surprisingly, the blood glucose in obese patients with Type 2 diabetes was well controlled without medication after surgery, and even cured completely [Carlsson, L. M. S., et al., Bariatric Surgery and Prevention of Type 2 Diabetes in Swedish Obese Subjects. New England Journal of Medicine, 2012. 367(8): p. 695-704, Schauer, P. R., et al., Bariatric surgery versus intensive medical therapy for diabetes—3-year outcomes. N Engl J Med, 2014. 370(21): p. 2002-13]. The drugs that simulate intestinal hormones, such as GLP-1 agonists and DPPIV inhibitors, have become oral hypoglycemic agents with highest prescription dose in the world, and related cardiovascular benefits have been reported.

The concept of enterotype [Arumugam, M., et al., Enterotypes of the human gut microbiome. Nature, 2011. 473 (7346): p. 174-80] was first proposed by Peer Bork, which meant that the composition of intestinal parasites were relatively fixed in the populations. There are 2 to 3 kinds of enterotypes in the populations. With the increased sample size and the improved sequencing technique, especially the promotion of the second generation of sequencing, the enterotype can be classified into 2 types: one is the Prevotella-based Prevotella enterotype, and the other is Bacteroides-based Bacteroides enterotype. At present, no evidence has shown the direct association between enterotype and various medical health indexes of human body. The corresponding gene function studies suggest the metabolic ability of vitamins is varied for different enterotypes, which is associated with the meat-vegetable dietary habit of the host. However, although gut microbiota is also considered to be an important medium of metabolism in human body [Haiser, H. J. and P. J. Turnbaugh, Is it time for a metagenomic basis of therapeutics? Science, 2012. 336(6086): p. 1253-5], no clinical trial evidences can be available now.

### SUMMARY OF INVENTION

An object of the present invention is to overcome the drawback of lack of directly relevant evidences between the enterotypes and health indicators of the human body in the prior art and provide characteristics of gut metagenome as a screening marker of Acarbose efficacy in patients with Type 2 diabetes; particularly provide an application of characteristics of gut microbiota metagenome as a screening marker of Acarbose efficacy in patients with Type 2 diabetes. In the present invention, it is discovered that patients with Type 2 diabetes with different gut microbiota showed significant difference in the therapeutic response to diabetic hypoglycemic agent-Acarbose. Therefore, the Bacteroides-based Bacteroides enterotype can be used as a screening marker of Acarbose efficacy in patients with Type 2 diabetes.

The object of the present invention is achieved through the following technical solutions:

The present invention relates to an application of characteristics of gut microbiota metagenome as a screening marker of Acarbose efficacy in patients with Type 2 diabetes, wherein the characteristics of gut microbiota metagenome is Bacteroides enterotype.

Preferably, the Bacteroides enterotype is determined by DNA sequencing or PCR amplification of parasites in feces in vitro.

Preferably, the PCR amplification specifically comprises: extract the DNA of parasites in feces in vitro and perform 16Sma PCR amplification for specific enrichment strains.

Preferably, the Bacteroides enterotype is determined by detecting secondary bile acid in the in vitro blood samples. The secondary bile acids include UDCA, TUDCA, GUDCA, DCA, TDCA, GDCA, LCA, TLCA, GLCA. In the present invention, two kinds of enterotypes are found, one is Prevotella-based Prevotella enterotype, and the other is Bacteroides-based Bacteroides enterotype. In the Bacteroides enterotype, the deoxycholic acid and lithocholic acid levels are significantly lower than those in Prevotella enterotype, while the ursodeoxycholic acid level with protective effect is higher than that in the Prevotella enterotype. The further gut metagenome analysis showed that, ursodesoxycholic acid is further decomposed into KO of lithocholic acid, which is apparently enriched in the Prevotella enterotype, suggesting that the metabolic ability of bile acids in gut microbiota was significantly different in patients with two enterotypes.

Preferably, the detection of secondary bile acid comprises the following steps:

S1. Sample pretreatment: Add 300 μL of internal standard methanol to every 75 μL of blood samples, to extract the target compound and precipitate the protein, vortex, centrifuge and draw the supernatant, then lyophilize, re-dissolve in 50 μL of acetonitrile solution (25%, volume), and wait for sample injection;

S2. Detection: conduct sample analysis using 1290 Infinity liquid phase and 6460A triple quadrupole mass spectrometry system;

Perform the liquid phase separation using 100 mm×2.1 mm ACQUITY UPLC C8 column having a particle size of 1.7 m, of which, phase A is 10 mM NH4HCO3 aqueous solution, phase B is pure acetonitrile; initially 25% phase B (by volume), retaining 0.5 min, followed by increased to 40% phase B (by volume) linearly within 12.5 min, then increased to 90% (by volume) within 1 min, flush the system for 3 min, recover to 25% phase B (by volume) in 0.5 min, after equilibrating 2.5 min, the flow rate is 0.35 ml/min, column temperature is 35° C. and the injection volume is 5 μL;

Mass spectrometry is performed by ESI source negative ion mode, with main parameters as follows: Gas Temp: 350° C.; Gas Flow: 8 l/min; Nebulizer: 40 psi; Sheath Gas Temp: 400° C.; Sheath Gas Flow: 8 l/min; Capillary: 3500 V; Nozzle voltage: 400 V.

Preferably, the efficacy of Acarbose in the patients with Type 2 diabetes and Bacteroides enterotype includes improving the insulin resistance, reducing the secondary bile acid, and promoting the reduction of cardiovascular risks in addition to glucose-lowering.

Preferably, the indicators for reducing the harmful secondary bile acid include GDCA, TDCA, TLCA, and the indicators for reducing the binding of taurine with bile acid include TCA, TDCA, TLCA, TUDCA.

Preferably, the indicators for improving insulin resistance include decreased fasting blood glucose, decreased fasting C peptide and insulin level, down-regulated waist-to-hip ratio, down-regulated HOMA insulin resistance index and up-regulated Adiponectin.

Preferably, the indicators that promote the reduction of cardiovascular risks include decreased PDGFAA, PDGFAABB, endothelin, and VegfC plasma factor.

The present invention further relates to a kit used for screening of Acarbose efficacy in patients with Type 2 diabetes, comprising:

A reagent used to collect in vitro stool samples or in vitro blood samples;

A reagent used to determine the enterotype by DNA sequencing or PCR amplification of the parasites in the in vitro stool samples, or a reagent used to determine the enterotype by detecting the secondary bile acid in the in vitro blood samples.

There are two kinds of enterotypes, one is Prevotella-based Prevotella enterotype, and the other is Bacteroides-based Bacteroides enterotype. Different enterotype can predict the benefits of patients for treatment of diabetes with Acarbose, especially the effect of improving insulin resistance, reducing secondary bile acid, and promoting the reduction of cardiovascular risks in addition to glucose-lowering. Specifically, Bacteroides enterotype has a better effect of improving insulin resistance, reducing secondary bile acid, and promoting the reduction of cardiovascular risks in addition to glucose-lowering.

Compared with prior art, the prevent invention can achieve the following beneficial effects:

1) It is discovered that patients with Type 2 diabetes with different gut microbiota showed significant difference in the therapeutic response to diabetic hypoglycemic agent-Acarbose by using the concept of enterotype firstly. Therefore, before medication, patients can be classified according to the enterotype, to select the populations with optimal efficacy and determine if an individual patient with Type 2 diabetes is applicable to Acarbose treatment.

2) The classification of enterotypes is generally based on DNA sequencing or PCR amplification of parasites in the feces; while in the baseline, the bile acid component, especially secondary bile acid, can be used for distinguishing the enterotypes; the typing of gut microbiota (i.e. enterotype) can be identified by the blood markers (i.e. secondary bile acid level in the plasma), to become a marker for diagnosis.

### BRIEF DESCRIPTION OF THE DRAWINGS

FIG. 1 is a schematic diagram of the typing of the enterotypes, of which, A. horizontal clustering and correlation of Bacteroides enterotype; B. horizontal clustering and correlation of Prevotella enterotype; C. Comparison of Bacteroides biological abundance in two enterotypes; D. Comparison of Prevotella biological abundance in two enterotypes. EntB: Bacteroides enterotype. EntP: Prevotella enterotype;

FIG. 2 shows the changes in curative effect indexes after treatment with Acarbose in two enterotypes, the black represents a significant decrease after treatment, white represents a significant increase after treatment, and gray represents no significant change after treatment, P<0.05;

FIG. 3 shows the difference of bile acid spectra and their changes after Acarbose treatment between the two enterotypes, of which, A. bile acids with difference in the baseline in two enterotypes; B. Difference of bile acid spectran between two enterotypes at baseline. C. Difference of two KO related to secondary bile acid metabolism between two enterotypes at baseline. D. The changed bile acid compositions and their signal maps between two enterotypes after Acarbose treatment. The black represents a significant decrease after treatment, white represents a significant increase after treatment, and gray represents no significant change after treatment, P<0.05.

### DETAILED DESCRIPTIONS OF THE INVENTION

The present invention will be described in detail with reference to the following embodiments. The following embodiments can help technicians skilled in the art to further understand this invention without limiting the invention in any way. It should be noted that a plurality of modifications and improvements may be made by those skilled in the art without departing from the spirit of the invention, all of which will fall within the scope of protection of the invention.

### Embodiment

For naïve patients with Type 2 diabetes, their liver and kidney functions, blood glucose and lipid levels, intestinal hormones, inflammatory factors, and cardiovascular risk-related factors are evaluated before medication. Their stool, urine, and blood samples are retained. After clear diagnosis and evaluation, patients are treated for 3 months at a daily dose of Acarbose 300 mg. The patients' blood glucose levels are followed up every month within 3 months, and the medication is adjusted according to the blood glucose level. Three months later, the pre-medication assessment is repeated, and the urine, stool and blood samples are retained.

Specific steps are as follows:

1. Collection of Clinical Samples

a) A randomized, opened, positive control method is adopted to collect the naïve patients with Type 2 diabetes and normal controls of their spouses. The clinical and biochemical data, gastrointestinal motility of diabetic patients before and after Acarbose treatment are compared and their blood and stool samples are collected. The newly diagnosed patients of Type 2 diabetes without medication receive routine examinations, including the retention of stool and blood samples.

Collection of Stool Samples

b) Stool

i. The instrument: Plastic basin (the diameter less than the caliber of household flush toilet)

ii. Freshness protection package, sterile small-handle spoon

iii. 50 ml sterile centrifuge tube

iv. Place the plastic basin (the diameter less than the caliber of household flush toilet) into a flush toilet, cover the freshness protection package (do not immerse the edges of the freshness protection package into the water of the toilet). If samples are taken in the hospital, cover a freshness protection package directly in the clean potty, to retain the stool samples;

v. Mix the upper layer of the fresh stool sample well with a small-handle spoon, pick up a small amount into a 50 ml sterile centrifuge tube. Take at least 10 g sample each tube, tighten the tube cover (indicate the sampling time, sampling group and number on the centrifuge tube wall). Retain stools for each subject each time, a total of 3 tubes of samples (RNAlater treatment tube, glycerine tube, and treatment-free tube).

vi. Immediately place the collected samples into −80° C. for cryopreservation.

c) Retention of Serum Samples

i. Draw venous blood 15 ml under fasting condition, of which, 1.5 ml is used for detection of plasma glucose, 6.5 ml is added to ordinary tubes (including aprotinin, DPPV inhibitor), and 6.5 ml is added to anticoagulant tubes (including heparin and RNA later)

ii. Centrifuge the blood in ordinary tube at 4° C., when serum is separated out, draw about 3 ml, and divide to three 1.5 ml Eppendorf on average, then tighten the tube caps;

iii. Centrifuge the anticoagulation blood immediately at 4° C., to separate out about 3 ml of plasma, then divide to three 1.5 ml imported RNAase free Eppendorf tubes on average,

iv. Indicate the sample name, center number and random number on the tube in details;

v. Cover the tubes for one week with plastic tapes;

vi. Keep them at −20° C. (placed at −80° C. if condition permitted), and subpackage the plasma and immediately place them at −20° C. or dry ice.

2. Determination of Bile Acid

The reagents Sodium taurochenodeoxycholate (TCDCA), Sodium glycocholate hydrate (GCA), Sodium taurodeoxycholate (TDCA), Chenodeoxycholic acid (CDCA), Ursodeoxycholic acid (UDCA), Taurocholic acid (TCA), Sodium glycodeoxycholate (GDCA), Glycoursodeoxycholic acid (GUDCA), Cholic acid (CA), Deoxycholic acid (DCA), Sodium glycochenodeoxycholate (GCDCA), Sodium tauroursodeoxycholate (TUDCA), Sodium taurolithocholate (TLCA), Lithocholic acid (LCA), NH₄HCO₃ are purchased from Sigma, USA; Glycochenodeoxycholic Acid 3-Sulfate Disodium Salt (GCDCS) are synthesized in the laboratory of Zhejiang University; Chenodeoxycholic Acid-d4 (CDCA-d4), Glycochenodeoxycholic Acid-d5 3-Sulfate Disodium Salt (GCDCS-d5), Taurochenodeoxycholic Acid-d5 (TCDCA-d5), Cholic Acid-d5(CA-d5), Glycocholic acid-d5(GCA-d5), Lithocholylglycine (GLCA) are purchased from TRC, Canada; and Taurodeoxycholic Acid-d5(TDCA-d5), Taurocholic acid-d5(TCA-d5) are purchased from CIL, USA. Acetonitrile and methanol are purchased from Merk, Germany.

Sample pretreatment: Take 75 μL of blood sample, add 300 μL of internal standard methanol, to extract the target compound and precipitate the protein, vortex 30s, centrifuge 10 min at the rate of 15000 rpm, draw 200 μL of the supernatant, then lyophilize, re-dissolve in 50 μL of 25% acetonitrile solution, and wait for sample injection. Instrument and method: conduct sample analysis using 1290 Infinity liquid phase (Agilent, USA) and 6460A triple quadrupole mass spectrometry system (Agilent, USA). Perform the liquid phase separation using 100 mm×2.1 mm ACQUITY UPLC C8 column having a particle size of 1.7 μm (Waters, USA), of which, phase A is 10 mM NH4HCO3 aqueous solution, phase B is pure acetonitrile; initially 25% phase B, retaining 0.5 min, followed by increased to 40% phase B linearly within 12.5 min, then increased to 90% within 1 min, flush the system for 3 min, recover to 25% phase B in 0.5 min, after equilibrating 2.5 min, the flow rate is 0.35 ml/min, column temperature is 35° C. and the injection volume is 5 μL; Mass spectrometry is performed by ESI source negative ion mode, with main parameters as follows: Gas Temp: 350° C.; Gas Flow: 8 l/min; Nebulizer: 40 psi; Sheath Gas Temp: 400° C.; Sheath Gas Flow: 8 l/min; Capillary: 3500 V; Nozzle voltage: 400 V. The bile acid is detected under a reaction monitoring mode (MRM). The concentration of the internal standard and the main mass spectrum parameters are shown in Table 1. The setting of mass spectrum parameters for bile acid analysis is shown in Table 2.

TABLE 1
Concentration of internal standard and main MS parameters

| | Con- | | | | Collision |
|---|---|---|---|---|---|
| | centration | parent | daughter | | Energy |
| | (mg/L) | ion | ion | Fragmentor | (eV) |
| CA-d5 | 0.08 | 412.5 | 412.5 | 200 | 10 |
| CDCA-d4 | 0.3 | 395.4 | 395.4 | 200 | 10 |
| GCA-d5 | 0.2 | 469.2 | 74.1 | 200 | 35 |
| GCDCA-d4 | 0.2 | 452.3 | 74.1 | 240 | 40 |
| GCDCS-d5 | 0.2 | 533.3 | 453.3 | 200 | 35 |
| TCA-d5 | 0.1 | 519.2 | 80.1 | 320 | 90 |
| TCDCA-d5 | 0.1 | 503.3 | 80.2 | 300 | 70 |
| TDCA-d5 | 0.1 | 503.3 | 80.2 | 300 | 70 |

TABLE 2
MS parameters for bile acid analysis and internal standard for calibration

| | Compound | Internal standard | parent ion | daughter ion | Fragmentor | Collision Energy eV |
|---|---|---|---|---|---|---|
| Lithocholic acid | LCA | CA-d5 | 375.3 | 375.3 | 200 | 2 |
| Chenodesoxycholic acid | CDCA | CDCA-d4 | 391.4 | 391.4 | 200 | 10 |
| Deoxycholic acid | DCA | CDCA-d4 | 391.4 | 391.4 | 200 | 10 |
| Ursodeoxycholic acid | UDCA | CDCA-d4 | 391.4 | 391.4 | 200 | 5 |
| Bile acid | CA | CA-d5 | 407.5 | 407.5 | 200 | 10 |
| Glycine conjugated with lithocholic acid | GLCA | GCA-d5 | 432.3 | 74 | 200 | 35 |
| Glycine conjugated with deoxycholic acid | GDCA | GCDCA-d4 | 448.2 | 74.1 | 200 | 40 |
| Glycine conjugated with chenodesoxycholic acid | GCDCA | GCDCA-d4 | 448.3 | 74.1 | 240 | 42 |
| Glycine conjugated with ursodeoxycholic acid | GUDCA | GCDCA-d4 | 448.3 | 74.1 | 200 | 40 |
| Glycine conjugated with bile acid | GCA | GCA-d5 | 464.2 | 74.1 | 200 | 35 |
| Taurine conjugated with lithocholic acid | TLCA | TCA-d5 | 482.1 | 80.1 | 300 | 70 |
| Taurine conjugated with chenodesoxycholic acid | TCDCA | TCDCA-d5 | 498.3 | 80.2 | 300 | 70 |
| Taurine conjugated with lithocholic acid | TDCA | TDCA-d5 | 498.3 | 80.2 | 300 | 70 |
| Taurine conjugated with ursodeoxycholic acid | TUDCA | TCA-d5 | 498.3 | 80.1 | 320 | 70 |
| Taurine conjugated with bile acid | TCA | TCA-d5 | 514.2 | 80.1 | 320 | 90 |

3. DNA Sequencing of Gut Microbiota

For HiSeq 2500 sequencing, the fragments at the length of 350 bp are used to establish the database and compare with 9.9M human intestinal gene set, to obtain the phylum, species and genus of IMG (70% coverage rate and 65% recognition rate at the phylum level, 85% recognition rate at the genus level, and 95% recognition rate at the species level). The clustering analysis of intestinal parasites is performed by principal component analysis (PCA).

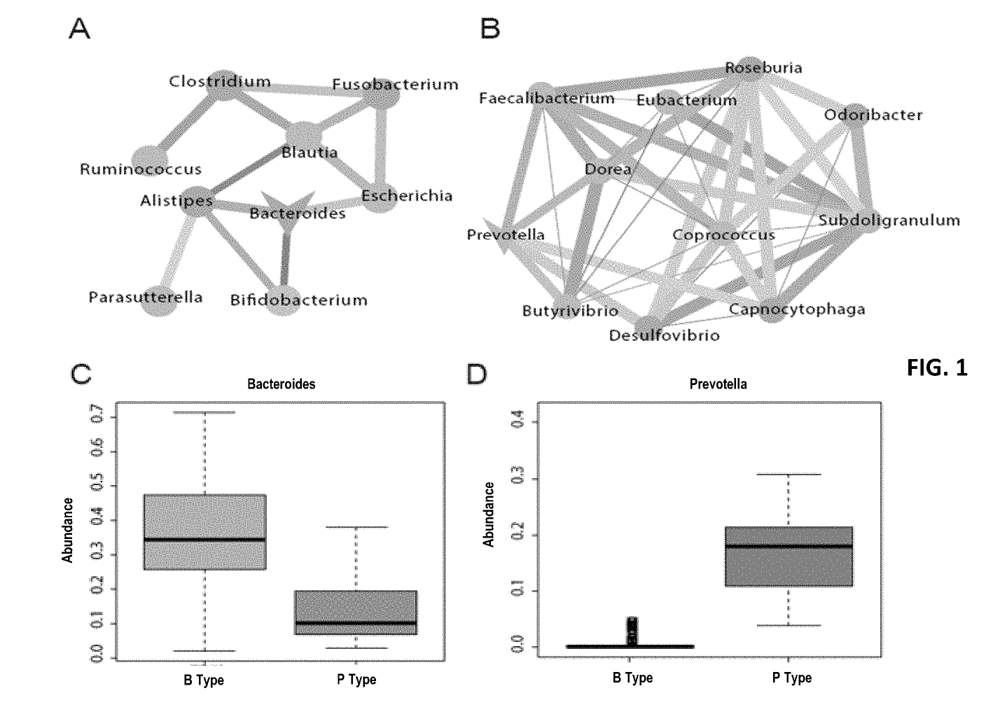

In the present invention, the gut microbiota colony DNA extraction and second generation sequencing of metagenome are performed in patients' feces, then compared with the published 9.9M human gut metagenome gene set, with a matching rate about 77%. About 143 kinds of gut microbiota with annotation information and difference before and after medication are found by clustering analysis. The genus level analysis shows that, different clustering of gut microbiota is found at the baseline in patients with Type 2 diabetes. According to the characteristic accumulation of gut microbiota, the enterotype is obtained. In the present invention, there are mainly two enterotypes: one is the Prevotella-based Prevotella enterotype, and the other is Bacteroides-based Bacteroides enterotype (FIG. 1).

According to enterotype classification, there are no significant differences in the sex and age distribution between the two types of patients with Type 2 diabetes. There are no significant differences in the baseline blood glucose levels, body weights, liver and kidney functions and other health indicators between them. Only the levels of red blood cells, hemoglobin and interleukin 6 are slightly higher in the patients of Prevotella enterotype (P<0.05). (Table 3)

After treatment, the efficacy of this drug for treatment of diabetes in the two groups of patients at baseline is observed.

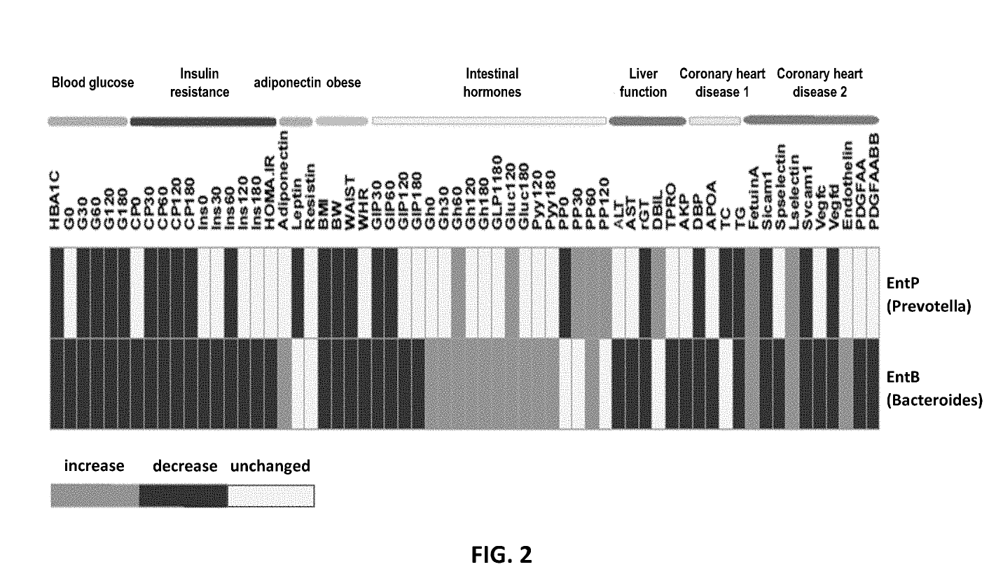

First, the most major efficacy of Acarbose is reflected by the reduction in 2h postprandial plasma glucose and HbA1c. But there are no differences in the two indexes between the two enterotypes (FIG. 2, Table 4).

Second, there is difference in control of fasting blood glucose level between the two enterotypes. The patients of Bacteroides enterotype show significant improvement and the degree of improvement after treatment; while the patients with Type 2 diabetes of the Prevotella enterotype do not show significant improvement in fasting blood glucose level. The other metabolic related indexes, including insulin, body weight, BMI, waist circumference, cardiovascular risk factors and intestinal hormones are significantly different between the two enterotypes.

The fasting C peptide and insulin levels are significantly decreased after treatment in the Bacteroides enterotype. After calculation, the HOMAIR index, which reflects insulin resistance, decreases after Acarbose treatment, but this benefit is significant only in the patients of Bacteroides enterotype, but not significant in patients of Prevotella enterotype, suggesting that patients with Type 2 diabetes of Bacteroides enterotype are more likely to improve their status of insulin resistance after taking Acarbose. The standard meal-induced insulin release curve, waist-to-hip ratio, and Adiponectin levels that are related to the insulin resistance show significant decrease after Acarbose treatment in T2DM patients of Bacteroides enterotype, but these indexes show no significant change in the patients of Prevotella enterotype.

Acarbose can cause a decrease in TG, APOA and DBP, and there are no significant differences between the two enterotypes after treatment; however, some plasma factors associated with diabetic vascular complications such as PDGFAA and PDGFAABB, endothelin, VegfC are significantly lower in the Bacteroides enterotype, suggesting that the Acarbose treatment can bring more benefits of reducing vascular complications in addition to lowering blood glucose level and risks of macrovascular diseases in the Bacteroides enterotype.

Gut hormone is a hot research target in the treatment of Type 2 diabetes, and its level is changed significantly in the Acarbose treatment. Among the several gut hormones detected, the elevated GLP1, glucagon, PYY, and ghrelin and GIP at each time point after medication are significant in the Bacteroides enterotype, but not significant in the Prevotella enterotype, suggesting that any metabolic benefit of Acarbose through gut hormones is more significant in the Bacteroides enterotype.

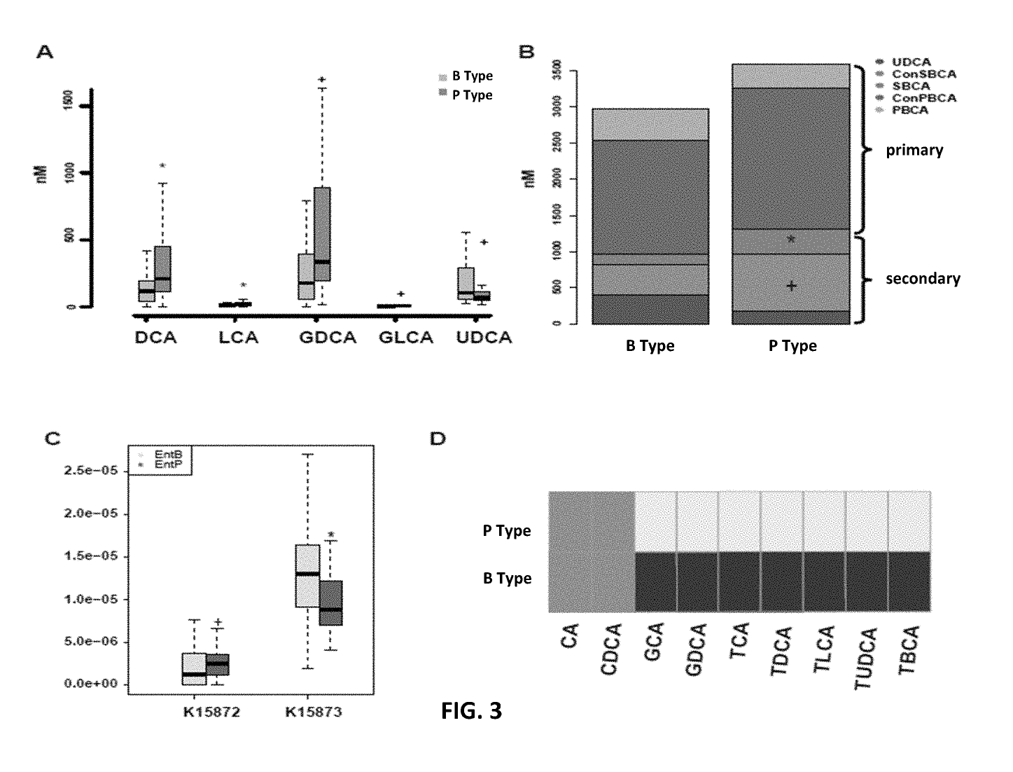

Third, there is a difference in bile acid spectrum between the two enterotypes at the baseline level (FIG. 3A, B). In the Bacteroides enterotype, the levels of deoxycholic acid and lithocholic acid in secondary bile acid are significantly lower than those in Prevotella enterotype, whereas the ursodeoxycholic acid level with protective effect is higher than that in Prevotella enterotype. The further gut metagenome analysis showed that, the ursodesoxycholic acid is further degraded into lithocholic acid KO, which is enriched in Prevotella enterotype apparently (FIG. 3C), suggesting that there is significant difference in the bile acid metabolic ability of gut microbiota between patients of two enterotypes. After treatment with Acarbose, the difference in bile acid composition is more pronounced between both enterotypes (FIG. 3D). The two kinds of primary bile acids are significantly increased in two enterotypes after treatment with Acarbose, but without enterotype-specific changes, suggesting that Acarbose may affect the whole reabsorption of bile acid in the small intestine.

Therefore, this study shows that, different enterotypes can predict patients' benefits from Acarbose treatment of diabetes, especially improving the insulin resistance, reducing the secondary bile acid, and promoting the reduction of cardiovascular risks in addition to glucose-lowering. The enterotype diagnosis can be completed by ordinary DNA PCR amplification of 16sRNA of characteristic bacteria genus, which is convenient and economical, making the precision medical care of Type 2 diabetes possible.

TABLE 3
Baseline clinical indexes of two enterotypes

| | | Bacteroides | Prevotella |
|---|---|---|---|
| Clinical Index | P value | enterotype | enterotype |
| SBP | 0.052305571 | 126.68 ± 15.68 | 134.92 ± 19.75 |
| DBP | 0.569158065 | 80.87 ± 8.49 | 82.54 ± 11.12 |
| Height | 0.406982243 | 166.76 ± 7.27 | 168.13 ± 7.97 |
| Body weight | 0.632776326 | 72.76 ± 9.93 | 74.44 ± 12.11 |
| BMI | 0.426864667 | 26.17 ± 3.48 | 26.2 ± 2.73 |
| Waist circumference | 0.382242756 | 90.58 ± 7.7 | 92.53 ± 10.05 |
| Hip circumference | 0.850454946 | 99.15 ± 7.58 | 98.71 ± 6.25 |
| Waist-to-hip ratio | 0.073022015 | 0.91 ± 0.05 | 0.94 ± 0.06 |
| RBC count | 0.008822773* | 4.77 ± 0.45 | 5.01 ± 0.3 |
| Hemoglobin | 0.015559629* | 142.47 ± 20.66 | 150.75 ± 11.96 |
| Hematocrit | 0.110126957 | 9.5 ± 22.96 | 9.69 ± 22.65 |
| WBC count | 0.221434929 | 6.35 ± 1.46 | 6.67 ± 1.35 |
| Granulocyte percentage | 0.721728164 | 57.18 ± 12.59 | 54.07 ± 17.36 |
| Percentage of | 0.823390429 | 31.2 ± 10.11 | 30.08 ± 12.05 |
| lymphocytes | | | |
| Percentage of | 0.92702484 | 5.68 ± 2.12 | 5.64 ± 2.2 |
| monocytes | | | |
| Platelet count | 0.977269042 | 207.14 ± 53.01 | 208.26 ± 53.28 |
| ALT | 0.770933665 | 34.74 ± 20.18 | 39.33 ± 38.6 |
| AST | 0.821446414 | 26.76 ± 12.39 | 32.45 ± 33.84 |
| Alkaline phosphatase | 0.003733751* | 68.02 ± 17.83 | 81.7 ± 21.67 |
| Glutamyl transpeptidase | 0.219732988 | 38.87 ± 37.46 | 48.13 ± 54.35 |
| Total bilirubin | 0.517075592 | 15.66 ± 5.85 | 14.33 ± 4.54 |
| Direct bilirubin | 0.930882347 | 3.17 ± 1.58 | 3.3 ± 1.57 |
| Total protein | 0.555631501 | 72.16 ± 4.18 | 72.99 ± 4.34 |
| Albumin | 0.439028944 | 46.81 ± 26.74 | 44.38 ± 4.21 |
| Uric acid | 0.389295382 | 5.01 ± 1.2 | 5.24 ± 1.03 |
| Creatinine | 0.424300196 | 66.58 ± 14.76 | 69.16 ± 12.41 |
| Urea nitrogen | 0.93814784 | 301.17 ± 73.87 | 301.29 ± 65.47 |
| Potassium | 0.802728704 | 6.46 ± 17.43 | 4.11 ± 0.51 |
| Sodium | 0.153693762 | 137.39 ± 17.91 | 140.64 ± 3.13 |
| Chloride | 0.45778794 | 102.73 ± 2.78 | 102.32 ± 2.5 |
| Triglycerides | 0.479383839 | 2.29 ± 1.56 | 2.28 ± 2.29 |
| Total cholesterol | 0.763815537 | 5.03 ± 0.91 | 5.05 ± 1.54 |
| High density lipoprotein | 0.974185078 | 3.05 ± 0.81 | 3.17 ± 1.05 |
| Low density lipoprotein | 0.341454604 | 1.2 ± 0.45 | 1.18 ± 0.2 |
| Apolipoprotein A | 0.99402696 | 1.34 ± 0.18 | 1.36 ± 0.17 |
| Apolipoprotein B | 0.923810467 | 1.03 ± 0.21 | 1.06 ± 0.29 |
| Lipoprotein A | 0.833966588 | 62.95 ± 144.85 | 164.75 ± 516.3 |
| Blood glucose 0 min | 0.091734172 | 7.91 ± 1.4 | 7.29 ± 1.11 |
| Blood glucose 30 min | 0.130269742 | 10.91 ± 1.96 | 10.27 ± 1.87 |
| Blood glucose 60 min | 0.257347624 | 14.24 ± 2.43 | 13.79 ± 1.84 |
| Blood glucose 120 min | 0.243421632 | 14.78 ± 2.86 | 14 ± 2.36 |
| Blood glucose 180 min | 0.741659488 | 12.11 ± 3.21 | 11.51 ± 3.04 |
| Serum insulin 0 min | 0.509699318 | 9.54 ± 4.74 | 11.83 ± 11.34 |
| Serum insulin 30 min | 0.234997693 | 19.7 ± 11.99 | 22.7 ± 12.31 |
| Serum insulin 60 min | 0.469868561 | 36.99 ± 21.41 | 42.04 ± 27.34 |
| Serum insulin 120 min | 0.517877918 | 48.41 ± 26.27 | 55.15 ± 35.47 |
| Serum insulin 180 min | 0.889080689 | 38.78 ± 26.07 | 35.42 ± 22.9 |
| Serum C-peptide 0 min | 0.979767063 | 2.5 ± 0.74 | 2.67 ± 1.27 |
| Serum C-peptide 30 min | 0.318552538 | 3.42 ± 1.1 | 3.6 ± 1.17 |
| Serum C-peptide 60 min | 0.694278947 | 5.08 ± 1.77 | 5.24 ± 1.86 |
| Serum C-peptide 120 min | 0.809629053 | 7.59 ± 2.29 | 7.62 ± 2.45 |
| Serum C-peptide 180 min | 0.343793846 | 7.3 ± 2.51 | 6.61 ± 1.97 |
| Plasma insulin 0 min | 0.469445501 | 478.88 ± 234.65 | 476.64 ± 338.58 |
| Plasma insulin 30 min | 0.810557675 | 791.99 ± 393.84 | 808.26 ± 506.26 |
| Plasma insulin 60 min | 0.838524751 | 1396.45 ± 752.85 | 1467.09 ± 1061.37 |
| Plasma insulin 120 min | 0.741764202 | 1934.72 ± 997.05 | 2110.35 ± 1443.51 |
| Plasma insulin 180 min | 0.754907581 | 1641.1 ± 947.84 | 1639.39 ± 1040.15 |
| Plasma C-peptide 0 min | 0.384853423 | 1175.79 ± 418.14 | 1127.3 ± 498.93 |
| Plasma C-peptide 30 min | 0.813883421 | 1670.96 ± 718.73 | 1669.2 ± 711.29 |
| Plasma C-peptide 60 min | 0.8047981 | 2578.14 ± 1156.59 | 2530 ± 1212.03 |
| Plasma C-peptide 120 min | 0.852096376 | 3871.17 ± 1525.71 | 3800.54 ± 1372 |
| Plasma C-peptide 180 min | 0.587827408 | 3626.21 ± 1473.21 | 3336.12 ± 1109.94 |
| HbAlc | 0.394975993 | 7.65 ± 0.91 | 7.45 ± 0.72 |
| GHRP 0 min | 0.647538837 | 47.52 ± 28.77 | 54.93 ± 51.33 |
| GHRP 30 min | 0.270618653 | 54.38 ± 33.75 | 51.8 ± 43.65 |
| GHRP 60 min | 0.100675425 | 45.72 ± 25.99 | 39.13 ± 30.64 |
| GHRP 120 min | 0.420950166 | 35.62 ± 17.43 | 34.83 ± 24.87 |
| GHRP 180 min | 0.608065141 | 42.79 ± 21.75 | 47.11 ± 36.95 |
| GLP-1 0 min | 0.925944614 | 6.46 ± 10.84 | 5.02 ± 5.17 |
| GLP-1 30 min | 0.874727025 | 19.49 ± 18.36 | 18.24 ± 15.26 |
| GLP-1 60 min | 0.891679993 | 16.79 ± 13.74 | 14.11 ± 8.46 |
| GLP-1 120 min | 0.706003564 | 11.35 ± 12.71 | 8.58 ± 7.07 |
| GLP-1 180 min | 0.412865834 | 9.46 ± 12.94 | 7.22 ± 6.79 |
| Glu 0 min | 0.976095762 | 29.11 ± 13.28 | 30.48 ± 17.95 |
| Glu 30 min | 0.738477919 | 37.97 ± 20.1 | 37.5 ± 23.81 |
| Glu 60 min | 0.605878342 | 35.31 ± 19.34 | 34.12 ± 23.53 |
| Glu 120 min | 0.343451344 | 25.45 ± 12.45 | 24.88 ± 16.59 |
| Glu 180 min | 0.911837296 | 22.34 ± 12.16 | 23.56 ± 14.08 |
| PYY 0 min | 0.902682115 | 53.23 ± 56.11 | 41.27 ± 30.44 |
| PYY 30 min | 0.826513742 | 67.21 ± 59.48 | 61.55 ± 43.07 |
| PYY 60 min | 0.984419278 | 64.4 ± 55.36 | 55.23 ± 32.99 |
| PYY 120 min | 0.556885888 | 55.41 ± 50.64 | 51.09 ± 30.71 |
| PYY 180 min | 0.705995014 | 57.51 ± 56.59 | 43.62 ± 32.17 |
| GIP 0 min | 0.466825554 | 76.52 ± 47.45 | 72.4 ± 53.13 |
| GIP 30 min | 0.959035633 | 358.7 ± 222.63 | 357.55 ± 244.17 |
| GIP 60 min | 0.693172872 | 443.67 ± 220.91 | 417.88 ± 238.84 |
| GIP 120 min | 0.453200572 | 406.03 ± 192.67 | 388.55 ± 218.63 |
| GIP 180 min | 0.181743927 | 323.22 ± 179.85 | 289.97 ± 235.54 |
| PP 0 min | 0.318610459 | 93.28 ± 78.66 | 82.39 ± 78.19 |
| PP 30 min | 0.569166173 | 313.48 ± 220.69 | 258.05 ± 114.37 |
| PP 60 min | 0.133570837 | 284.41 ± 196.22 | 217.63 ± 115.08 |
| PP 120 min | 0.245241771 | 222.38 ± 163.09 | 180.86 ± 108.39 |
| PP 180 min | 0.106766755 | 180.24 ± 111.6 | 143.88 ± 97.4 |
| IL 17M | 0.962918479 | 138.85 ± 74.04 | 155.04 ± 123.32 |
| G-CSF | 0.803059249 | 50.43 ± 21.81 | 49.25 ± 17.99 |
| IFN r1 | 0.181566671 | 45.55 ± 42.56 | 34.79 ± 17.12 |
| IL 10 | 0.861536003 | 1.68 ± 1.51 | 1.69 ± 1.65 |
| MIP-3a | 0.333709066 | 22.75 ± 19.47 | 25.06 ± 16.77 |
| IL 12 | 0.993168795 | 8.76 ± 7.01 | 8.42 ± 5.75 |
| IL 15 | 0.267007222 | 232.97 ± 132.57 | 258.05 ± 119.82 |
| IL 17A | 0.7415298 | 32.16 ± 9.43 | 31.08 ± 9.36 |
| IL 22 | 0.13451041 | 42.34 ± 40.57 | 30.96 ± 10.48 |
| IL 9 | 0.480277271 | 81.35 ± 54.9 | 69.56 ± 35.58 |
| IL 33 | 0.751136369 | 37.46 ± 13.13 | 35.71 ± 13.65 |
| IL 2 | 0.472384797 | 58.54 ± 49.48 | 47.94 ± 29.24 |
| IL 21 | 0.905751857 | 28.21 ± 11.92 | 31.75 ± 22.21 |
| IL 4 | 0.927445079 | 20.29 ± 15.95 | 19.9 ± 13.78 |
| IL 23 | 0.902434691 | 37.04 ± 23.49 | 36.37 ± 20.38 |
| IL 5 | 0.892399481 | 45.18 ± 36.22 | 38.48 ± 19.04 |
| IL IL17E | 0.608790357 | 25.15 ± 31.08 | 19.56 ± 10.22 |
| IL 27 | 0.534356146 | 71.75 ± 56.63 | 60.5 ± 37.44 |
| IL 31 | 0.793285936 | 112.57 ± 57.24 | 101.87 ± 34.87 |
| TNF B | 0.885718446 | 45.25 ± 32.83 | 40.52 ± 18.34 |
| IL 28a | 0.591202712 | 20.21 ± 9.34 | 19.56 ± 8.97 |
| FGF 19M | 0.802617256 | 23.01 ± 4.13 | 23.27 ± 3.78 |
| FGF 23M | 0.132019218 | 45.88 ± 9.26 | 41.96 ± 6.75 |
| Oncostatin | 0.496200994 | 105.9 ± 75.52 | 93.87 ± 65.35 |
| cTn | 0.139027346 | 19.77 ± 18.76 | 14.9 ± 10.08 |
| ET | 0.560717515 | 12.79 ± 2.7 | 12.88 ± 3.41 |
| FGF 21 | 0.738341043 | 0.3 ± 0.32 | 0.26 ± 0.26 |
| NGF | 0.100733692 | 2.41 ± 3.11 | 2.83 ± 2.61 |
| HGF | 0.271836439 | 408.2 ± 258.41 | 465.78 ± 249 |
| MCP 1 | 0.454428023 | 244.24 ± 136.45 | 246.4 ± 77.85 |
| TNF a | 0.292633798 | 4.56 ± 2.19 | 5.67 ± 4.46 |
| SICAM | 0.684945545 | 161.33 ± 75.77 | 188.08 ± 166.69 |
| MPO | 0.27368969 | 385.65 ± 351.89 | 464.48 ± 363.39 |
| sP-selectin | 0.165666585 | 141.01 ± 76.01 | 270.22 ± 572.86 |
| sVCAM | 0.233334305 | 703.18 ± 244.75 | 820.42 ± 410.18 |
| PDGF AA | 0.945399512 | 4951.68 ± 1904.72 | 4902.38 ± 1831.41 |
| PDGF AABB | 0.490732791 | 26581.76 ± 14551.83 | 27347.81 ± 11562.13 |
| RANTES | 0.899107284 | 54893.62 ± 24860.48 | 52830.46 ± 19005.15 |
| LEP | 0.632956992 | 5307140.26 ± 3052569.78 | 6121581.85 ± 4297113.91 |
| NGAL | 0.43181483 | 136543.91 ± 75938.84 | 158878.69 ± 102095.95 |
| Resistin | 0.051869812 | 16813.35 ± 9816.98 | 21730.58 ± 11698.65 |
| Adipokine | 0.377073702 | 3322727.99 ± 1554037.78 | 3526181.62 ± 1630515.78 |
| PAI 1 | 0.635961359 | 57833.47 ± 21242.95 | 59240 ± 19939.5 |
| CRP | 0.310428088 | 11.92 ± 15.92 | 21.22 ± 49.48 |
| FETU-A | 0.790035938 | 333.82 ± 50.16 | 332.62 ± 40.54 |
| L-selectin | 0.973025194 | 1.54 ± 0.25 | 1.58 ± 0.32 |
| FABP 3 | 0.706810959 | 2282.3 ± 1074.01 | 2481.19 ± 1192.16 |
| FABP 4 | 0.706016525 | 14084.38 ± 12064.5 | 13559.88 ± 12861.95 |
| ECGF | 0.1033731 | 187.05 ± 127.4 | 234.75 ± 131.23 |
| ET | 0.640239461 | 3.78 ± 1.99 | 4.08 ± 3.37 |
| FGF 1 | 0.400574693 | 13.71 ± 10.83 | 12.86 ± 5.24 |
| VEGF c | 0.614322703 | 113.88 ± 37.89 | 113.02 ± 46.82 |
| VEGF d | 0.115360982 | 163.15 ± 117.25 | 203.19 ± 123.58 |
| FGF 2 | 0.685128912 | 37.29 ± 22.25 | 41.01 ± 26.94 |
| VEGF a | 0.416512674 | 499.98 ± 398.81 | 400.25 ± 267.47 |
| LEP | 0.732125461 | 6490.77 ± 6637.01 | 6502.46 ± 5785.56 |
| IL 8 | 0.812673509 | 17.03 ± 26.21 | 18.69 ± 24.86 |
| IL 1b | 0.912521353 | 0.48 ± 0.79 | 0.59 ± 1.29 |
| IL 13 | 0.442815325 | 237.23 ± 132.1 | 254.36 ± 129.07 |
| IL 6 | 0.044391053* | 2.85 ± 5.27 | 3.1 ± 3.63 |
| LBP | 0.185062045 | 20.61 ± 7.28 | 18.66 ± 7.99 |
| HOMA-IR | 0.895763831 | 3.33 ± 1.73 | 3.83 ± 3.38 |
| Plasma HOMA-IR | 0.206702804 | 3.76 ± 2.07 | 3.44 ± 2.46 |

TABLE 4

| | P value | | Mean | | | |
|---|---|---|---|---|---|---|
| | Bacterioide | Prevotella | Acarbose | | | |
| | enterotype | enterotype | Bacterioide enterotype | | Prevotella enterotype | |
| | Acarbose | Acarbose | Baseline | After treatment | Baseline | After treatment |
| Adipisin | 0.735655998 | 0.678771973 | 3168874.417 | 3174901.441 | 3105379.8 | 3221619.467 |
| Adpn* | 0.009070051 | 0.072998047 | 4659142.417 | 5157339.824 | 6825515.733 | 10063264.07 |
| AKP* | 0.000298293 | 0.148678549 | 68.38235294 | 59.22857143 | 85.42666667 | 77.5 |
| ALB | 0.052938425 | 0.125506471 | 49.74545455 | 42.26 | 45.58571429 | 44.38571429 |
| ALT | 0.023923875 | 0.315055201 | 37.68 | 29.42857143 | 40.7 | 44.35714286 |
| APOA* | 0.011314861 | 0.125 | 1.328214286 | 1.2475 | 1.377142857 | 1.267272727 |
| APOB | 0.42397348 | 0.1875 | 1.029642857 | 0.963571429 | 1.131428571 | 0.927272727 |
| AST* | 0.009259021 | 0.183967151 | 28.20294118 | 22.45714286 | 35.91333333 | 29.35714286 |
| BMI* | 3.26E−05 | 0.003494192 | 26.42555556 | 25.51138889 | 26.06866667 | 25.11333333 |
| bun | 0.017056015 | 0.221189047 | 309.2823529 | 329.2 | 306.212 | 326.4615385 |
| BW* | 2.32E−05* | 0.00367322* | 74.45277778 | 71.91111111 | 75.38 | 72.63333333 |
| cl | 0.365775393 | 0.149551339 | 102.9375 | 103.4 | 101.6923077 | 103.3571429 |
| CP0* | 0.000751479 | 0.229309082 | 2.620833333 | 2.216388889 | 2.668666667 | 2.485333333 |
| CP120* | 5.92E−08 | 0.001159668 | 7.842777778 | 5.204166667 | 7.615333333 | 5.304666667 |
| CP180* | 1.25E−09 | 0.012036948 | 7.327777778 | 4.843333333 | 6.262666667 | 4.734666667 |
| CP30* | 3.59E−05 | 0.023068064 | 3.602222222 | 2.7525 | 3.502 | 2.802 |
| CP60* | 5.42E−06 | 6.10E−05 | 5.375 | 3.71 | 5.294 | 3.609333333 |
| creatine | 0.530974718 | 1 | 67.10882353 | 67.61428571 | 67.98666667 | 67.41538462 |
| crp | 0.852740904 | 0.229309082 | 13.5725 | 29.96117647 | 10.216 | 19.638 |
| DBIL | 0.42621214 | 0.833885439 | 2.755882353 | 2.965714286 | 3.542857143 | 6.292857143 |
| DBP* | 0.000195001 | 0.049089037 | 82.08333333 | 75.37142857 | 80.46666667 | 74.93333333 |
| egf | 0.458006509 | 0.267578125 | 193.7005882 | 183.3530303 | 206.3742857 | 267.604 |
| ENDOM | 0.030053592 | 0.207824804 | 12.36111111 | 13.43939394 | 13.73333333 | 14.26666667 |
| Endothelin* | 0.039909487 | 0.363606971 | 3.46 | 4.158571429 | 4.926428571 | 4.776 |
| FABP3 | 0.903465115 | 0.890380859 | 2374.760833 | 2350.673429 | 2293.333333 | 2146.933333 |
| FABP4 | 0.103153911 | 0.252380371 | 14146.31429 | 11504.54286 | 12673.33333 | 10510.4 |
| fetuinA* | 0.000150225 | 0.000610352 | 340.4180556 | 400.9188235 | 334.9993333 | 417.3386667 |
| fgf1 | 0.327136563 | 0.777511253 | 11.08944444 | 11.65771429 | 14.00642857 | 13.78933333 |
| FGF19M | 0.000721153 | 0.006268599 | 22.44444444 | 19.73529412 | 22.6 | 19.3 |
| FGF1M | 0.327494344 | 0.62856768 | 21.94444444 | 22.57352941 | 24.4 | 24.33333333 |
| fgf2 | 0.271079158 | 0.94425068 | 33.13027778 | 35.39542857 | 42.55357143 | 43.98266667 |
| fgf21 | 0.502579383 | 0.779828433 | 0.317777778 | 0.37 | 0.339285714 | 0.296 |
| fgf21M | 0.000200362 | 0.002624512 | 143.0416667 | 100.0588235 | 136.4 | 97.93333333 |
| FGF23M | 5.55E−05 | 0.628914718 | 44.90277778 | 37.07352941 | 42.36666667 | 43.46666667 |
| FGF2M | 0.219893155 | 0.805893176 | 12.30555556 | 12.69117647 | 13.13333333 | 13.66666667 |
| G120* | 1.75E−07 | 6.10E−05 | 14.64277778 | 9.171388889 | 13.292 | 9.170666667 |
| G180* | 7.36E−09 | 0.000244141 | 11.68388889 | 7.941388889 | 10.65 | 7.852857143 |
| G30* | 4.07E−10 | 0.00012207 | 10.73055556 | 7.622777778 | 9.652 | 7.601428571 |
| G60* | 5.82E−11 | 0.00012207 | 13.99611111 | 9.074166667 | 13.12066667 | 9.055 |
| GIP0 | 0.890667984 | 0.71484375 | 79.71542857 | 80.26388889 | 84.04533333 | 57.39785714 |
| GIP120* | 0.007692964 | 0.067626953 | 382.0711765 | 264.0158333 | 354.922 | 227.7985714 |
| GIP180* | 0.002999473 | 0.067626953 | 290.1938235 | 201.5647222 | 259.104 | 194.8235714 |
| GIP30* | 0.01013191 | 0.020263672 | 336.1434286 | 219.1305556 | 342.732 | 208.4314286 |
| GIP60* | 0.000305745 | 0.00402832 | 439.1731429 | 258.945 | 382.3233333 | 217.2485714 |
| GMCSFM | 0.400942689 | 0.798033799 | 49.375 | 64.61764706 | 55.3 | 67.96666667 |
| GO* | 2.34E−06 | 0.30279541 | 7.9075 | 6.606944444 | 7.002666667 | 6.712 |
| HB | 0.866145989 | 0.063711823 | 144.9714286 | 140.6431429 | 152.3333333 | 147.8333333 |
| HBA1C* | 3.54E−07 | 0.001087736 | 7.625 | 6.425 | 7.306666667 | 6.313333333 |
| HDL | 0.888463907 | 0.109863281 | 2.97 | 2.989411765 | 3.304615385 | 2.868571429 |
| HGF | 0.162910502 | 0.561401367 | 415.1552778 | 434.1691176 | 377.43 | 400.8593333 |
| HIP | 0.135245032 | 0.9372558 | 100.2028571 | 98.82777778 | 98.06666667 | 97.21428571 |
| HOMA.IR | 6.62E−05 | 0.638671875 | 3.585236667 | 2.487479506 | 3.79496563 | 3.326864889 |
| IFNr1 | 0.259007015 | 0.609162891 | 38.76388889 | 42.10294118 | 37.6 | 38.8 |
| IL10 | 0.241252899 | 0.635498047 | 1.525384615 | 2.300869565 | 1.787857143 | 1.797692308 |
| IL12 | 0.444698256 | 0.524475098 | 7.881142857 | 10.11903226 | 9.138666667 | 9.944 |
| IL13 | 0.394072711 | 0.735351563 | 231.2214286 | 311.6553846 | 251.2146667 | 279.3569231 |
| IL15M | 0.098387918 | 0.488708496 | 224.3034286 | 333.31875 | 269.8993333 | 291.8106667 |
| IL17AM | 0.823963715 | 0.726607536 | 32.86111111 | 33.73529412 | 33.2 | 32.03333333 |
| IL17EM | 0.870855906 | 0.949882816 | 21.51388889 | 22.23529412 | 21.36666667 | 21.23333333 |
| IL17FM | 0.055283514 | 0.30279541 | 124.9583333 | 181.3529412 | 183.1333333 | 232.6333333 |
| IL1B | 0.694494109 | 1 | 0.374722222 | 0.532352941 | 0.324285714 | 0.910666667 |
| IL1bM | 0.359000687 | 0.977243513 | 13.77777778 | 14.66176471 | 12.53333333 | 22.23333333 |
| IL21 | 0.850725867 | 0.394055106 | 26.04166667 | 29.13235294 | 35.9 | 34.46666667 |
| IL22M | 0.175108876 | 0.191177717 | 36.47222222 | 42.5 | 32.83333333 | 30.5 |
| IL23M | 0.085674334 | 0.460211098 | 34.80555556 | 39.35294118 | 39.56666667 | 41.7 |
| IL27 | 0.81743512 | 0.348590881 | 59.43055556 | 61.27941176 | 69.36666667 | 73.1 |
| IL28a | 0.80929836 | 0.18707508 | 20.02777778 | 20.04411765 | 20.9 | 18.36666667 |
| IL2M | 0.567407854 | 0.267970259 | 45.19444444 | 53.45588235 | 53.2 | 58.76666667 |
| IL31M | 0.270120255 | 0.719726563 | 98.86111111 | 112.9558824 | 105.7333333 | 112.3333333 |
| IL33M | 0.304945974 | 0.181618578 | 36.18055556 | 42.13235294 | 38.7 | 43.5 |
| IL4M | 0.104184122 | 0.120544434 | 17.69057143 | 30.73387097 | 22.642 | 31.074 |
| IL5M | 0.644309509 | 0.890380859 | 34.56944444 | 38.44117647 | 42.33333333 | 43.96666667 |
| IL6 | 0.346414942 | 0.12890625 | 2.577586207 | 4.845555556 | 1.631111111 | 2.665 |
| IL6M | 0.724177037 | 0.04439591 | 42.08333333 | 55.85294118 | 31.06666667 | 40.53333333 |
| IL8 | 0.648417992 | 0.463134766 | 15.06722222 | 11.92176471 | 9.640714286 | 15.43866667 |
| IL9M | 0.644339761 | 0.488708496 | 67.23611111 | 84.63235294 | 76.13333333 | 84.9 |
| INS0 | 0.000815737 | 0.798233177 | 10.30666667 | 8.284444444 | 12.27 | 11.734 |
| INS120 | 2.86E−06 | 0.003356934 | 50.66222222 | 30.33111111 | 59.02866667 | 27.83866667 |
| INS180 | 1.30E−06 | 0.02557373 | 39.57222222 | 22.83277778 | 35.00266667 | 20.88266667 |
| INS30 | 0.000414037 | 0.018066406 | 22.1 | 14.49833333 | 22.598 | 14.20533333 |
| INS60 | 0.000114652 | 0.004272461 | 40.315 | 23.70222222 | 45.708 | 23.404 |
| K | 0.665771961 | 0.239135782 | 4.2628125 | 7.892571429 | 4.193846154 | 4.054285714 |
| LBP | 0.317407376 | 0.390991211 | 19.56027778 | 17.36264706 | 15.45214286 | 17.616 |
| LDL | 0.122171856 | 0.556146527 | 1.154285714 | 1.241764706 | 1.201538462 | 1.168571429 |
| LEPTIN | 0.157752971 | 0.006713867 | 6318.866667 | 6480.940882 | 7200.680714 | 4521.537333 |
| LPA | 0.433978558 | 0.625 | 66.63321429 | 32.841 | 303.14 | 214.7509091 |
| Lselectin* | 0.034002222 | 0.003438 | 1.522777778 | 1.686176471 | 1.591333333 | 2.047333333 |
| Lymph | 0.873712631 | 0.030175493 | 30.11470588 | 28.87428571 | 32.56785714 | 28.21428571 |
| MCP1 | 0.069589183 | 0.006713867 | 245.4563889 | 256.6388235 | 211.976 | 252.7773333 |
| MIP3A | 0.000646785 | 0.100000618 | 20.46548387 | 43.76967742 | 20.34642857 | 29.93571429 |
| monocyte | 0.810992023 | 0.861220365 | 5.728125 | 5.480571429 | 5.481538462 | 5.828571429 |
| mpo | 0.542937988 | 1 | 366.3775 | 275.7567647 | 411.0126667 | 467.2593333 |
| Na | 0.097485128 | 0.064957971 | 139.40625 | 136.8574286 | 140.3076923 | 142.2142857 |
| NEU | 0.279685889 | 0.001656248 | 60.20588235 | 58.46857143 | 52.50571429 | 63.59285714 |
| NGAL | 0.787128593 | 0.276855469 | 129328.8056 | 125535.0294 | 137207.4667 | 174248.3333 |
| NGF | 0.78915379 | 0.887042291 | 1.8775 | 1.865 | 3.097333333 | 2.434 |
| oscatinM | 0.231878957 | 0.454284668 | 105.3888889 | 86.93939394 | 85.43333333 | 101.1 |
| PAI1 | 0.7103313 | 0.252380371 | 56209.66667 | 52769.94118 | 54084.73333 | 61006.73333 |
| pCP0 | 0.003281006 | 0.583007813 | 1264.89 | 1098.934444 | 1066.730667 | 1065.952857 |
| pCP120 | 3.16E−05 | 0.00012207 | 3983.970588 | 2787.309722 | 3804.866667 | 2723.04 |
| pCP180 | 1.50E−06 | 0.024536133 | 3659.411765 | 2451.553333 | 3335.466667 | 2526.179286 |
| pCP30 | 4.56E−05 | 0.013427734 | 1799.170286 | 1394.301389 | 1597.980667 | 1362.325714 |
| pCP60 | 0.000122547 | 0.000610352 | 2780.398571 | 1891.040833 | 2546.807333 | 1935.42 |
| PDGFAA | 0.046347912 | 0.488708496 | 5322.730571 | 4701.948824 | 4845.333333 | 4507.333333 |
| PDGFAABB | 0.004710246 | 0.561401367 | 29869.68571 | 24130.63636 | 26779.73333 | 24006.8 |
| pgh0* | 0.009070051 | 0.206054688 | 42.28705882 | 55.75416667 | 50.14833333 | 53.4025 |
| pgh0180* | 0.002696353 | 0.16015625 | 41.34516129 | 60.30111111 | 46.26363636 | 50.87333333 |
| pgh120* | 3.87E−06 | 0.07421875 | 34.24242424 | 58.55861111 | 35.18454545 | 42.5275 |
| pgh30* | 0.000781945 | 0.233398438 | 46.99676471 | 70.15138889 | 44.22846154 | 64.28307692 |
| pgh60* | 8.13E−05 | 0.041311226 | 43.87 | 66.50916667 | 33.40307692 | 56.35083333 |
| pGLP10 | 0.31839608 | 0.577148438 | 4.098333333 | 4.564117647 | 5.125 | 3.643846154 |
| pGLP1120 | 0.066918263 | 0.855224609 | 7.805294118 | 10.47416667 | 8.146666667 | 9.07 |
| pGLP1180* | 0.012499022 | 0.501586914 | 5.6478125 | 7.518333333 | 6.400666667 | 6.457857143 |
| pGLP130 | 0.062569209 | 0.057373047 | 15.29647059 | 12.20628571 | 20.41714286 | 11.40714286 |
| pGLP160 | 0.642244034 | 0.807739258 | 14.34941176 | 13.20333333 | 14.42066667 | 13.72 |
| pgluc0 | 0.234643616 | 0.637695313 | 29.12342857 | 37.29794118 | 31.76666667 | 39.20090909 |
| pgluc120* | 0.00154797 | 0.02734375 | 24.03272727 | 35.94735294 | 23.82571429 | 29.62545455 |
| pgluc180* | 0.000424966 | 0.153576397 | 21.732 | 32.29147059 | 23.65714286 | 30.42818182 |
| pgluc30 | 0.385465678 | 0.909667969 | 38.742 | 43.28277778 | 37.34333333 | 41.17166667 |
| pgluc60 | 0.016413683 | 0.518554688 | 35.55685714 | 43.96361111 | 30.278 | 36.68 |
| pHOMA.IR* | 0.00102708 | 0.71484375 | 3.951110809 | 2.565193754 | 3.249134038 | 2.716974268 |
| pins0* | 0.009625357 | 0.71484375 | 494.0097143 | 391.0080556 | 469.0473333 | 416.3028571 |
| pins120* | 3.16E−05 | 0.057373047 | 2011.773529 | 1168.609722 | 2303.542 | 1373.493077 |
| pins180* | 7.24E−06 | 0.067626953 | 1613.704118 | 916.6516667 | 1780.701333 | 1152.767143 |
| pins30* | 0.003007197 | 0.172607422 | 817.7231429 | 561.7958333 | 780.2073333 | 639.8921429 |
| pins60* | 0.000283359 | 0.03527832 | 1469.366571 | 892.8875 | 1574.948667 | 968.9964286 |
| platelet | 0.964365002 | 0.670003472 | 212.1714286 | 213.9714286 | 208.8 | 206.2666667 |
| PP0 | 0.727960433 | 0.049438477 | 85.98828571 | 80.31583333 | 107.5466667 | 69.67214286 |
| PP120 | 0.050439826 | 0.03527832 | 231.4273529 | 258.8033333 | 203.484 | 260.1364286 |
| PP180 | 0.446560021 | 0.501586914 | 182.5308824 | 173.9941667 | 171.2373333 | 204.6585714 |
| PP30 | 0.703771093 | 0.03527832 | 319.3008571 | 331.6930556 | 279.108 | 376.6992857 |
| PP60 | 0.045642266 | 0.016601563 | 305.7042857 | 330.7513889 | 237.3526667 | 318.92 |
| ppyy0 | 0.539026541 | 0.91015625 | 42.186 | 49.602 | 51.0175 | 43.56636364 |
| ppyy120* | 0.038631681 | 0.921875 | 44.34516129 | 54.44848485 | 61.04916667 | 54.01538462 |
| ppyy180* | 0.033681393 | 0.431640625 | 45.31344828 | 56.15371429 | 54.65916667 | 51.59230769 |
| ppyy30 | 0.707845747 | 0.359375 | 56.97933333 | 55.73625 | 77.28307692 | 64.33909091 |
| ppyy60 | 0.33367527 | 0.764648438 | 56.174375 | 59.37029412 | 69.93692308 | 56.77923077 |
| RANTES | 0.179124002 | 0.524475098 | 58731.5 | 50811.91176 | 50815.2 | 47562.53333 |
| RBC | 0.321296464 | 0.306429005 | 4.783428571 | 4.832 | 5.008 | 4.945333333 |
| RCv | | 0.942925705 | 9.936176471 | 2.580285714 | 9.274 | 0.442142857 |
| resistin | 0.624272656 | 0.276855469 | 15960.5 | 14639.64706 | 21493.73333 | 25013.66667 |
| rGT* | 5.29E−05 | 0.020263672 | 42.75714286 | 24.82285714 | 60.07333333 | 43.12142857 |
| SBP | 0.122812744 | 0.064531987 | 127.0277778 | 120.8611111 | 127.8 | 119.8 |
| sicam1* | 2.21E−06 | 0.018066406 | 167.5533333 | 112.2894118 | 163.1493333 | 122.9993333 |
| Spselectin* | 0.001039933 | 0.120544434 | 142.3733333 | 92.83058824 | 139.506 | 102.7933333 |
| svcam1* | 0.000430483 | 0.041259766 | 674.3169444 | 484.8473529 | 738.3453333 | 545.498 |
| TBIL | 0.316992017 | 0.216308594 | 15.36176471 | 16.35714286 | 13.74285714 | 14.9 |
| TC | 0.059412126 | 0.026855469 | 4.968857143 | 4.735588235 | 5.514615385 | 4.882857143 |
| TG* | 0.000665817 | 0.01668677 | 2.482857143 | 1.525 | 2.532307692 | 1.693571429 |
| TNFa | 0.877708881 | 0.07823218 | 4.660833333 | 4.772647059 | 3.962666667 | 4.572 |
| TNFBM | 0.572530012 | 0.900061308 | 35.625 | 38.44117647 | 45.33333333 | 44.7 |
| TPRO* | 0.031620353 | 0.57587042 | 72.07941176 | 70.11428571 | 74.08571429 | 73.55 |
| Trophonin M | 0.796523868 | 0.167835191 | 17.48611111 | 16.15151515 | 16.53333333 | 17.9 |
| urea | 0.205820381 | 0.700616425 | 4.894848485 | 4.731428571 | 5.081333333 | 4.953846154 |
| vegfa | 0.253948028 | 0.172607422 | 516.5967647 | 475.2033333 | 308.4314286 | 340.1686667 |
| vegfc | 0.025448661 | 0.414306641 | 121.5308824 | 106.3214706 | 113.8453846 | 128.6914286 |
| vegfd | 9.05E−06 | 0.010742188 | 193.5427778 | 145.3523529 | 249.5521429 | 195.0753333 |
| WAIST* | 0.004813593 | 0.029960994 | 91.11428571 | 88.45277778 | 92.26666667 | 88.92857143 |
| WBC | 0.387914524 | 0.334179383 | 6.413428571 | 6.912571429 | 6.38 | 6.146 |
| WHR* | 0.036332936 | 0.068270928 | 0.909714286 | 0.895555556 | 0.94 | 0.913571429 |

What is claimed is:

1. An application of characteristics of gut microbiota metagenome as a as a screening marker of Acarbose efficacy in patients with Type 2 diabetes, wherein the characteristic of gut microbiota metagenome is Bacteroides enterotype.

2. The application according to claim 1, wherein the Bacteroides enterotype is determined by DNA sequencing or PCR amplification of parasites in feces in vitro.

3. The application according to claim 2, wherein the PCR amplification specifically comprises: extract the DNA of parasites in feces in vitro and perform 16Srna PCR amplification for specific enrichment strains.

4. The application according to claim 1, wherein the Bacteroides enterotype is determined by detecting secondary bile acid in the in vitro blood samples.

5. The application according to claim 4, wherein the detection of secondary bile acid comprises the following steps:
S1. Sample pretreatment: Add 300 μL of internal standard methanol to every 75 μL of blood samples, to extract the target compound and precipitate the protein, vortex, centrifuge and draw the supernatant, then lyophilize, re-dissolve in 50 μL of acetonitrile solution (25%, volume), and wait for sample injection;
S2. Detection: conduct sample analysis using 1290 Infinity liquid phase and 6460A triple quadrupole mass spectrometry system;
Perform the liquid phase separation using 100 mm×2.1 mm ACQUITY UPLC C8 column having a particle size of 1.7 μm, of which, phase A is 10 mM NH4HCO3 aqueous solution, phase B is pure acetonitrile; initially 25% phase B (by volume), retaining 0.5 min, followed by increased to 40% phase B (by volume) linearly within 12.5 min, then increased to 90% (by volume) within 1 min, flush the system for 3 min, recover to 25% phase B (by volume) in 0.5 min, after equilibrating 2.5 min, the flow rate is 0.35 ml/min, column temperature is 35° C. and the injection volume is 5 μL;
Mass spectrometry is performed by ESI source negative ion mode, with main parameters as follows: Gas Temp: 350° C.; Gas Flow: 8 l/min; Nebulizer: 40 psi; Sheath Gas Temp: 400° C.; Sheath Gas Flow: 8 l/min; Capillary: 3500 V; Nozzle voltage: 400 V.

6. The application according to claim 1, wherein the efficacy of Acarbose in the patients with Type 2 diabetes and Bacteroides enterotype includes improving the insulin resistance, reducing the secondary bile acid, and promoting the reduction of cardiovascular risks in addition to glucose-lowering.

7. The application according to claim 6, wherein the indicators for reducing the secondary bile acid include GDCA, TDCA, TLCA, and the indicators for reducing the binding of taurine with bile acid include TCA, TDCA, TLCA, TUDCA.

8. The application according to claim 6, wherein the indicators for improving insulin resistance include decreased fasting blood glucose, decreased fasting C peptide and insulin level, down-regulated waist-to-hip ratio, down-regulated HOMA insulin resistance index and up-regulated Adiponectin.

9. The application according to claim 6, wherein the indicators that promote the reduction of cardiovascular risks include decreased PDGFAA, PDGFAABB, endothelin, and VegfC plasma factor.

10. A kit used for screening of Acarbose efficacy in patients with Type 2 diabetes, comprising:
a reagent used to collect in vitro stool samples or in vitro blood samples; and
a reagent used to determine the enterotype by DNA sequencing or PCR amplification of the parasites in the in vitro stool samples, or a reagent used to determine the enterotype by detecting the secondary bile acid in the in vitro blood samples.
